(19) United States
(12) Patent Application Publication
(10) Pub. No.: US 2022/0213167 A1
(43) Pub. Date: Jul. 7, 2022

(54) ENGINEERED CELLS EXPRESSING ANTI-TUMOR T CELL RECEPTORS AND METHODS OF USE THEREOF

(71) Applicant: GigaMune, Inc., South San Francisco, CA (US)

(72) Inventors: Matthew James Spindler, San Francisco, CA (US); David Scott Johnson, San Francisco, CA (US); Adam Shultz Adler, Belmont, CA (US); Michael Asensio, South San Francisco, CA (US)

(21) Appl. No.: 17/608,261

(22) PCT Filed: May 1, 2020
(86) PCT No.: PCT/US2020/031018

§ 371 Date: Nov. 2, 2021

Related U.S. Application Data
(60) Provisional application No. 62/842,691, filed on May 3, 2019.

(51) Int. Cl. C07K 14/725; C12N 5/0783

(52) U.S. Cl. CPC C07K 14/7051; C12N 5/0636

(57) ABSTRACT

Provided herein are compositions comprising recombinant mammalian cells that express recombinant T cell receptors with specificity against gp100 peptide:MHC antigens. Also provided are therapeutic methods of using the recombinant mammalian cells as cell therapies against melanoma tumors.

### 1. CROSS REFERENCE TO RELATED APPLICATIONS

This application claims priority to U.S. Provisional Application No. 62/842,691, filed May 3, 2019 including the sequence listing filed with the application as a text file; the contents are incorporated by reference for all purposes.

### 2. SEQUENCE LISTING

The instant application contains a Sequence Listing which is submitted as an ASCII file on duplicate CDs labeled Copy 1 and Copy 2 and mailed on May 1, 2020 via USPS Priority Mail Express and is hereby incorporated by reference in its entirety. Said ASCII file, created on Apr. 30, 2020, is named GGM-003WOsequencelisting.txt, and is 213 MB in size.

### 3. FIELD

Provided herein are T cell receptors (TCRs) with binding specificity for gp100, also known as premelanosome protein (PMEL), and compositions comprising such TCRs, including non-natural DNA vectors encoding TCRs, pharmaceutical compositions, and non-natural cell therapies.

### 4. BACKGROUND

Melanoma, also known as malignant melanoma, is a type of cancer that develops from the pigment-containing cells known as melanocytes. Melanomas typically occur in the skin, but may rarely occur in the mouth, intestines, or eye. In women, they most commonly occur on the legs, while in men they are most common on the back. Sometimes they develop from a mole with changes such as an increase in size, irregular edges, change in color, itchiness, or skin breakdown.

The primary cause of melanoma is ultraviolet light (UV) exposure in those with low levels of skin pigment. The UV light may be from either the sun or from other sources, such as tanning devices. About 25% develop from moles. Those with many moles, a history of affected family members, and who have poor immune function are at greater risk. A number of rare genetic defects such as xeroderma pigmentosum also increase risk. Diagnosis is by biopsy and analysis of any skin lesion that has signs of being potentially cancerous.

Melanoma is the most dangerous type of skin cancer. Globally, in 2012, it newly occurred in 232,000 people. In 2015 there were 3.1 million with active disease which resulted in 59,800 deaths. Australia and New Zealand have the highest rates of melanoma in the world. There are also high rates in Northern Europe and North America, while it is less common in Asia, Africa, and Latin America. Melanoma is more common in men than women. Melanoma has become more common since the 1960s in areas which are mostly populated with white people.

Immunotherapy is aimed at stimulating the person's immune system against the tumor, by enhancing the body's own ability to recognize and kill cancer cells. The current approach to treating melanoma with immunotherapy includes three broad categories of treatments including cytokines, immune check point inhibitors, and adoptive cell transfer. These treatment options are most often used in people with metastatic melanoma and significantly improves overall survival.

Adoptive cell therapy (ACT) using tumor-infiltrating lymphocytes (TILs) isolated from a person's own melanoma tumor. These cells are grown in large numbers in a laboratory and returned to the patient after a treatment that temporarily reduces normal T cells in the patient's body. TIL therapy following lymphodepletion can result in durable complete response in a variety of setups.

The second treatment, adoptive transfer of genetically altered autologous lymphocytes, depends on delivering genes that encode so called T cell receptors (TCRs), into patient's lymphocytes. After that manipulation lymphocytes recognize and bind to certain molecules found on the surface of melanoma cells and kill them.

### 5. SUMMARY

Provided herein are novel TCRs with binding specificity for gp100, also known as premelanosome protein (PMEL), a protein highly expressed in melanoma and other tumors. Specifically, these TCRs target gp100 peptides presented by major histocompatibility complex (pMHC).

Also provided are isolated polynucleotides encoding the TCRs provided herein, and portions thereof.

Also provided are vectors comprising such polynucleotides.

Also provided are recombinant host cells comprising such polynucleotides and recombinant host cells comprising such vectors.

Also provided are methods of producing the TCRs using the polynucleotides, vectors, or host cells provided herein.

Also provided are pharmaceutical compositions comprising the TCRs and a pharmaceutically acceptable excipient.

In some aspects, the present invention provides a pharmaceutical composition comprising the TCR and an excipient. In some embodiments, the TCR is in an amount sufficient as prophylaxis against melanoma or other tumor when administered to a subject. In some embodiments, the TCR is an amount sufficient to clear melanoma or other tumor in an individual actively fighting disease.

In other aspects, the present invention provides a method of treating a disease comprising the step of: administering an effective amount of the TCR or the pharmaceutical composition provided herein to a subject with the disease.

In some aspects, the present invention provides a mixture of polynucleotides encoding the TCRs provided herein. In other aspects, the present invention provides a mixture of vectors comprising the isolated polynucleotides. In other aspects, the present invention provides a mixture of host cell clones comprising the mixture of polynucleotides or vectors.

Some aspects of the present invention are related to a method of producing TCR, comprising: expressing the antibodies in host cells using a library of polynucleotide vectors, and isolating the cells that express the TCR.

TCRs of the invention may be transformed into T cells, rendering them capable of destroying cells presenting gp100 pMHC, such as melanoma tumor cells, for administration to a patient in the treatment process known as adoptive therapy (see Zhao et al., (2007) J Immunol. 179: 5845-54; Robbins et al., (2008) J Immunol. 180: 6 116-31; and WO2008/038002).

In other aspects, the present invention provides methods for discovery of TCRs from highly diverse mammalian T cell repertoires.

### 6. BRIEF DESCRIPTION OF THE DRAWINGS

FIG. 1 summarizes the method of discovering TCRs from transcripts expressed in peripheral blood TCRs isolated from virus seropositive human donors.

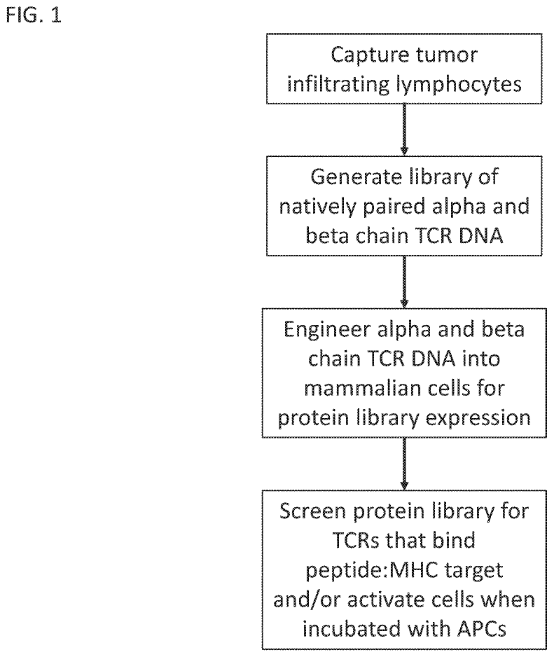

FIG. 2 summarizes a method of encapsulating T cells into physical containers with lysis mix and solid supports that capture nucleic acid targets from lysed cells.

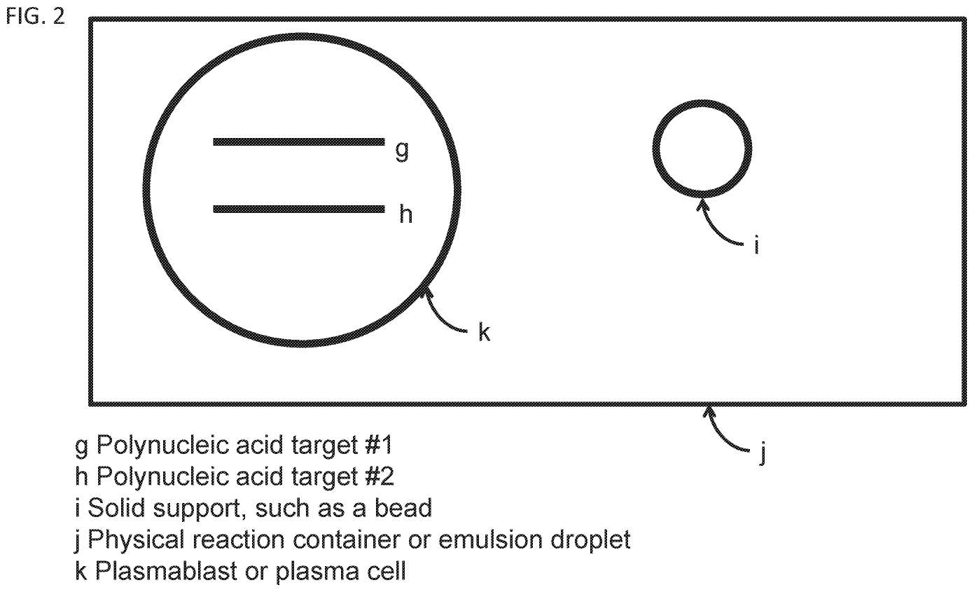

FIG. 3 summarizes a method of encapsulating target-specific primers with nucleic acid targets affixed to solid supports.

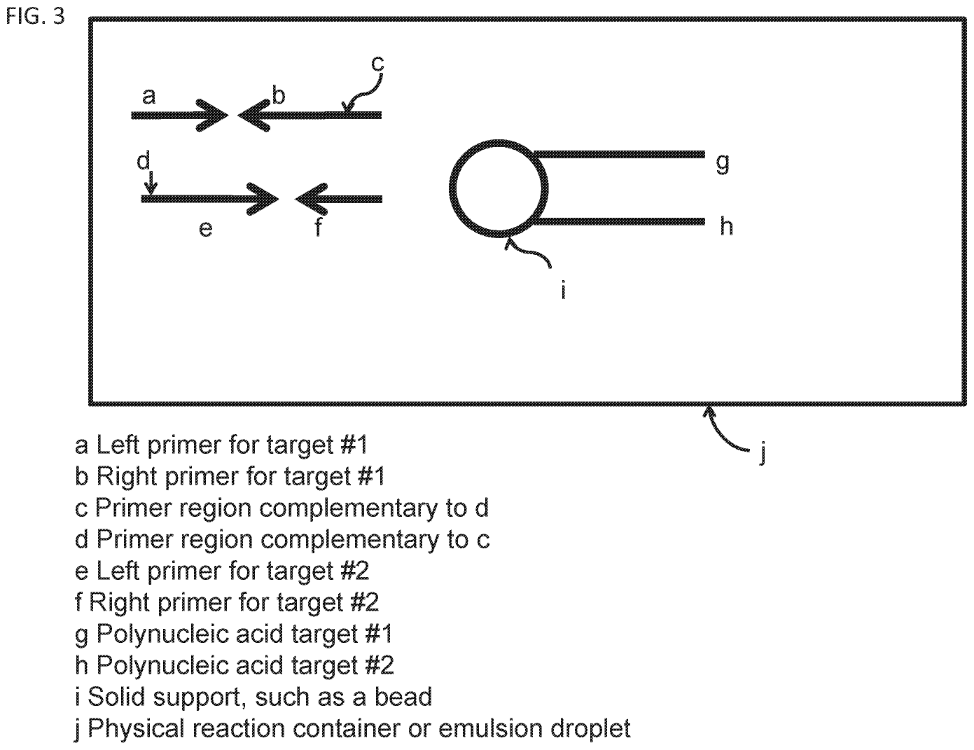

FIG. 4 shows the method of amplifying individual target nucleic acids with complementary regions.

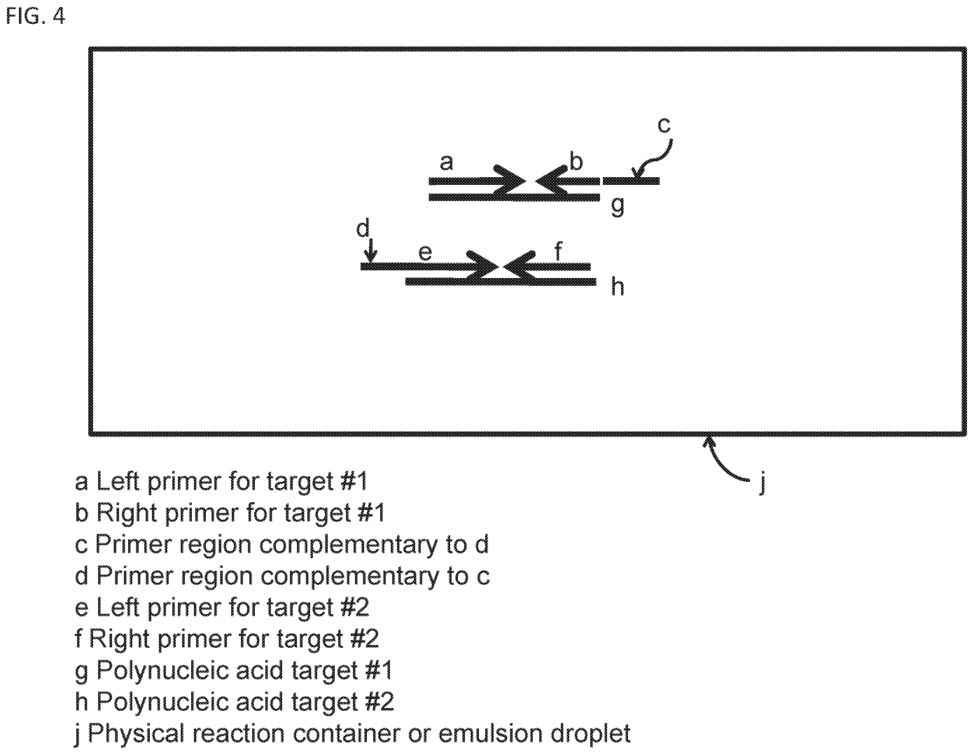

FIG. 5 shows the individual amplified target nucleic acids with complementary regions.

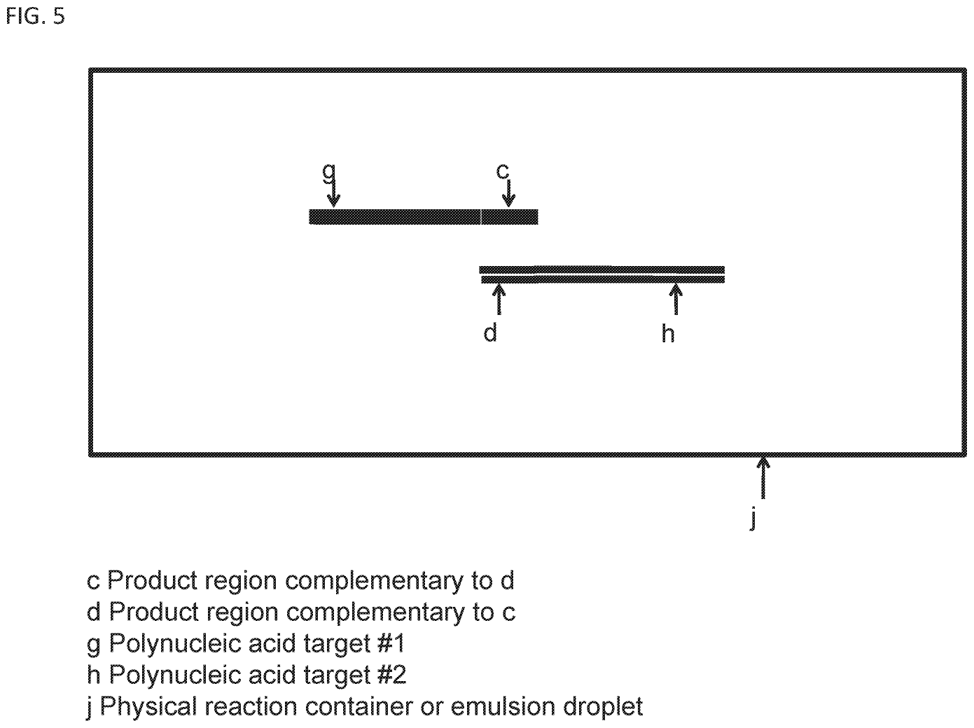

FIG. 6 summarizes a method of fusing separate amplified nucleic acid targets into single fused nucleic acid constructs.

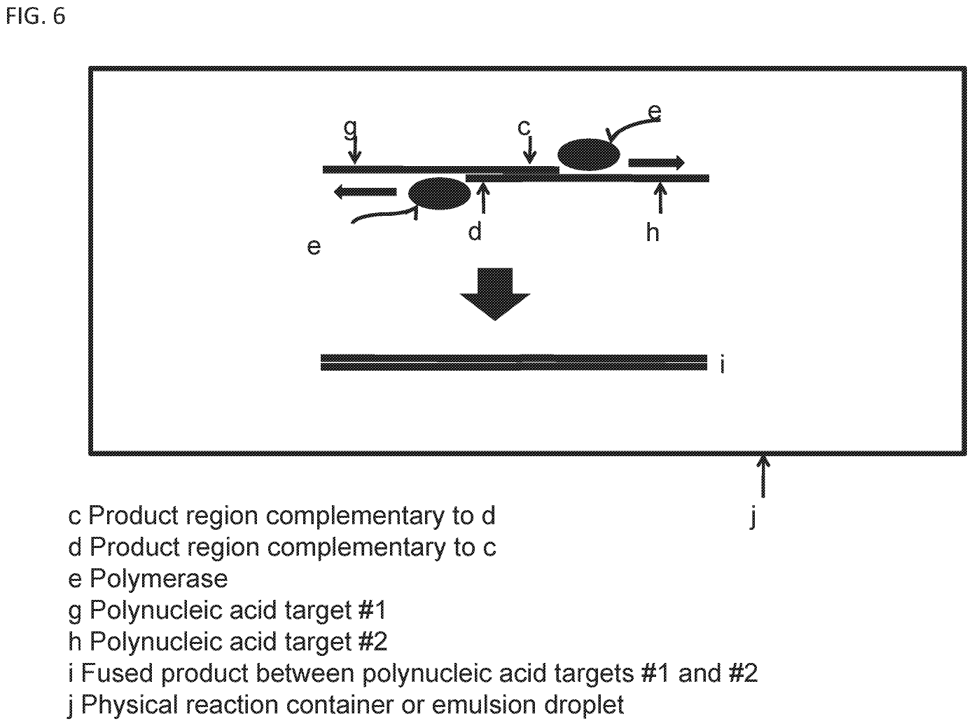

FIG. 7 shows the method of generating circularized gene expression constructs from the fused nucleic acid constructs.

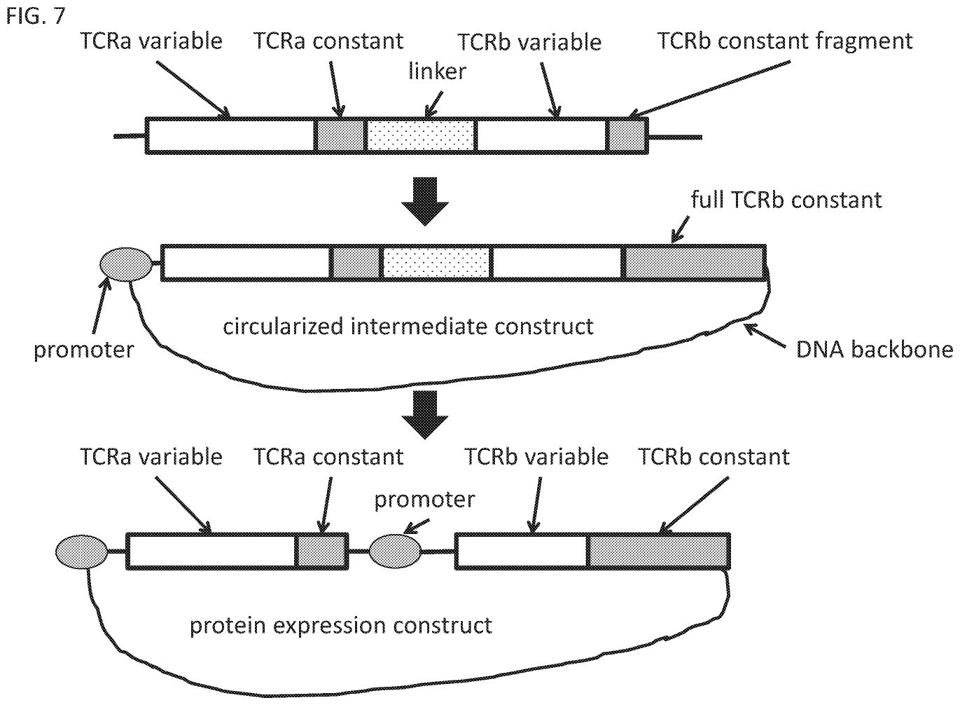

### 7. DETAILED DESCRIPTION

### 8. Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients.

The following terms, unless otherwise indicated, shall be understood to have the following meanings:

The term “T cell receptor”, or “TCR”, is a molecule found on the surface of T cells, or T lymphocytes, that is responsible for recognizing fragments of antigen as peptides bound to major histocompatibility complex (MHC) molecules. The TCR is composed of two different protein chains (that is, it is a heterodimer). In humans, in 95% of T cells the TCR consists of an alpha (α) chain and a beta (β) chain (encoded by TRA and TRB, respectively), whereas in 5% of T cells the TCR consists of gamma and delta (γ/δ) chains (encoded by TRG and TRD, respectively). This ratio changes during ontogeny and in diseased states (such as leukemia). It also differs between species. Each locus can produce a variety of polypeptides with constant and variable regions. When the TCR engages with antigenic peptide and MHC (peptide:MHC), the T lymphocyte is activated through signal transduction, that is, a series of biochemical events mediated by associated enzymes, co-receptors, specialized adaptor molecules, and activated or released transcription factors. The TCR is a disulfide-linked membrane-anchored heterodimeric protein normally consisting of the highly variable alpha (α) and beta (β) chains expressed as part of a complex with the invariant CD3 chain molecules. T cells expressing this receptor are referred to as α:β (or αβ) T cells, though a minority of T cells express an alternate receptor, formed by variable gamma (γ) and delta (δ) chains, referred as γδ T cells. Each chain is composed of two extracellular domains: Variable (V) region and a Constant (C) region, both of Immunoglobulin superfamily (IgSF) domain forming antiparallel β-sheets. The Constant region is proximal to the cell membrane, followed by a transmembrane region and a short cytoplasmic tail, while the Variable region binds to the peptide/MHC complex. The variable domain of both the TCR α-chain and β-chain each have three hypervariable or complementarity determining regions (CDRs). There is also an additional area of hypervariability on the β-chain (HV4) that does not normally contact antigen and, therefore, is not considered a CDR. The residues in these variable domains are located in two regions of the TCR, at the interface of the α- and β-chains and in the β-chain framework region that is thought to be in proximity to the CD3 signal-transduction complex. CDR3 is the main CDR responsible for recognizing processed antigen, although CDR1 of the alpha chain has also been shown to interact with the N-terminal part of the antigenic peptide, whereas CDR1 of the β-chain interacts with the C-terminal part of the peptide. CDR2 is thought to recognize the MHC. CDR4 of the β-chain is not thought to participate in antigen recognition. The constant domain of the TCR consists of short connecting sequences in which a cysteine residue forms disulfide bonds, which form a link between the two chains. The generation of TCR diversity is similar to that for antibodies and B cell antigen receptors. It arises mainly from genetic recombination of the DNA encoded segments in individual somatic T cells by somatic V(D)J recombination using RAG1 and RAG2 recombinases. Unlike immunoglobulins, TCR genes do not undergo somatic hypermutation. Each recombined TCR possess unique antigen specificity, determined by the structure of the antigen-binding site formed by the α and β chains in case of αβ T cells or γ and δ chains on case of γδ T cells.

“Affinity” refers to the strength of the sum total of non-covalent interactions between a single binding site of a molecule (e.g., an RPP) and its binding partner (e.g., an antigen or epitope). Unless indicated otherwise, as used herein, “affinity” refers to intrinsic binding affinity, which reflects a 1:1 interaction between members of a binding pair (e.g., TCR and peptide:MHC). The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein. Affinity can be determined, for example, using surface plasmon resonance (SPR) technology (e.g., BIACORE) or biolayer interferometry (e.g., FORTEBIO®).

“Avidity” refers to the accumulated strength of multiple affinities of individual non-covalent binding interactions, such as between a protein receptor and its ligand, and is commonly referred to as functional affinity. As such, avidity is distinct from affinity, which describes the strength of a single interaction. However, because individual binding events increase the likelihood of other interactions to occur (i.e. increase the local concentration of each binding partner in proximity to the binding site), avidity should not be thought of as the mere sum of its constituent affinities but as the combined effect of all affinities participating in the biomolecular interaction.

The “major histocompatibility complex” (MHC) is a set of cell surface proteins essential for the acquired immune system to recognize foreign molecules in vertebrates, which in turn determines histocompatibility. The main function of MHC molecules is to bind to antigens derived from pathogens and display them on the cell surface for recognition by the appropriate T-cells. The MHC determines compatibility of donors for organ transplant, as well as one's susceptibility to an autoimmune disease via crossreacting immunization. The human MHC is also called the HLA (human leukocyte antigen) complex (often just the HLA).

“MHC class I” molecules are one of two primary classes of MHC molecules and are found on the cell surface of all nucleated cells in the bodies of jawed vertebrates. They also occur on platelets, but not on red blood cells. Their function is to display peptide fragments of proteins from within the cell to cytotoxic T cells, often termed “peptide:MHC”; this will trigger an immediate response from the immune system against a particular non-self antigen displayed with the help of an MHC class I protein. Because MHC class I molecules present peptides derived from cytosolic proteins, the pathway of MHC class I presentation is often called cytosolic or endogenous pathway.

With regard to the binding of an TCR to a target peptide:MHC, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the RPP to the target molecule is competitively inhibited by the control molecule.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution an amino acid with a chemically or functionally similar amino acid. Conservative substitution tables providing similar amino acids are well known in the art. By way of example, the groups of amino acids provided in TABLES 1-3 are, in some embodiments, considered conservative substitutions for one another.

TABLE 1
Selected groups of amino acids that are considered conservative
substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 2
Additional selected groups of amino acids that are considered
conservative substitutions for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 3
Further selected groups of amino acids that are considered
conservative substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, N.Y. An RPP generated by making one or more conservative substitutions of amino acid residues in a parent RPP is referred to as a “conservatively modified variant.”

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminish of any direct or indirect pathological consequences of the disease, preventing metastasis, decreasing the rate of disease progression, amelioration or palliation of the disease state, and remission or improved prognosis.

As used herein, the term “therapeutically effective amount” or “effective amount” refers to an amount of an RPP or pharmaceutical composition provided herein that, when administered to a subject, is effective to treat a disease or disorder.

As used herein, the term “subject” means a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with an RPP provided herein. In some aspects, the disease or condition is a cancer.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

A “chemotherapeutic agent” refers to a chemical compound useful in the treatment of cancer. Chemotherapeutic agents include “anti-hormonal agents” or “endocrine therapeutics” which act to regulate, reduce, block, or inhibit the effects of hormones that can promote the growth of cancer.

The term “pharmaceutical composition” refers to a preparation which is in such form as to permit the biological activity of an active ingredient contained therein to be effective in treating a subject, and which contains no additional components which are unacceptably toxic to the subject.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “effector T cell” includes T helper (i.e., CD4+) cells and cytotoxic (i.e., CD8+) T cells. CD4+ effector T cells contribute to the development of several immunologic processes, including maturation of B cells into plasma cells and memory B cells, and activation of cytotoxic T cells and macrophages. CD8+ effector T cells destroy virus-infected cells and tumor cells. See Seder and Ahmed, Nature Immunol., 2003, 4:835-842, incorporated by reference in its entirety, for additional information on effector T cells.

The term “regulatory T cell” includes cells that regulate immunological tolerance, for example, by suppressing effector T cells. In some aspects, the regulatory T cell has a CD4+CD25+Foxp3+ phenotype. In some aspects, the regulatory T cell has a CD8+CD25+ phenotype. See Nocentini et al., Br. J. Pharmacol., 2012, 165:2089-2099, incorporated by reference in its entirety, for additional information on regulatory T cells.

A “cytotoxic T cell” (also known as TC, cytotoxic T lymphocyte, CTL, T-killer cell, cytolytic T cell, CD8+ T-cell or killer T cell) is a T lymphocyte (a type of white blood cell) that kills cancer cells, cells that are infected (particularly with viruses), or cells that are damaged in other ways. Most cytotoxic T cells express T-cell receptors (TCRs) that can recognize a specific antigen. An antigen is a molecule capable of stimulating an immune response, and is often produced by cancer cells or viruses. Antigens inside a cell are bound to class I MHC molecules, and brought to the surface of the cell by the class I MHC molecule, where they can be recognized by the T cell. If the TCR is specific for that antigen, it binds to the complex of the class I MHC molecule and the antigen, and the T cell destroys the cell.

The term “in vivo” translates to “in the living”, and refers to scientific studies in which the effects of various biological entities are tested on whole, living organisms or cells, usually animals, including humans, and plants, as opposed to a tissue extract or dead organism. This is not to be confused with experiments done in vitro (“within the glass”), i.e., in a laboratory environment using test tubes, Petri dishes, etc. Examples of investigations in vivo include: the pathogenesis of disease by comparing the effects of bacterial infection with the effects of purified bacterial toxins; the development of non-antibiotics, antiviral drugs, and new drugs generally; and new surgical procedures. Consequently, animal testing and clinical trials are major elements of in vivo research. In vivo testing is often employed over in vitro because it is better suited for observing the overall effects of an experiment on a living subject.

The term “recombinant” refers to proteins that result from the expression of recombinant DNA within living cells. Recombinant DNA is the general name for a piece of DNA that has been created by the combination of at least two separate segments of DNA.

The term “in vitro” translates to “in the glass”, and refers to scientific studies that are performed with microorganisms, cells, or biological molecules outside their normal biological context. Colloquially called “test-tube experiments”, these studies in biology and its subdisciplines are traditionally done in labware such as test tubes, flasks, Petri dishes, and microtiter plates. Studies conducted using components of an organism that have been isolated from their usual biological surroundings permit a more detailed or more convenient analysis than can be done with whole organisms; however, results obtained from in vitro experiments may not fully or accurately predict the effects on a whole organism. In contrast to in vitro experiments, in vivo studies are those conducted in animals, including humans, and whole plants.

A “variant” of a polypeptide (e.g., a TCR) comprises an amino acid sequence wherein one or more amino acid residues are inserted into, deleted from and/or substituted into the amino acid sequence relative to the native polypeptide sequence, and retains essentially the same biological activity as the native polypeptide. The biological activity of the polypeptide can be measured using standard techniques in the art (for example, if the variant is an TCR, its activity may be tested by binding assays, as described herein). Variants of the invention include fragments, analogs, recombinant polypeptides, synthetic polypeptides, and/or fusion proteins.

A “derivative” of a polypeptide is a polypeptide (e.g., a TCR) that has been chemically modified, e.g., via conjugation to another chemical moiety such as, for example, polyethylene glycol, albumin (e.g., human serum albumin), phosphorylation, and glycosylation. Unless otherwise indicated, the term “TCR” includes, in addition to antibodies comprising two full-length TCR alpha and two full-length TCR beta chains, derivatives, variants, fragments, and muteins thereof, examples of which are described below.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06.

A “host cell” is a cell that can be used to express a nucleic acid, e.g., a nucleic acid of the invention. Typically, a host cell is a cultured cell that can be transformed or transfected with a polypeptide-encoding nucleic acid, which can then be expressed in the host cell.

The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but to the progeny or potential progeny of such a cell. Because certain modifications may occur in succeeding generations due to, e.g., mutation or environmental influence, such progeny may not, in fact, be identical to the parent cell, but are still included within the scope of the term as used herein.

“Cell therapy” (also called cellular therapy or cytotherapy) is therapy in which cellular material is injected, grafted or implanted into a patient; this generally means intact, living cells. For example, T cells capable of fighting cancer cells via cell-mediated immunity may be injected in the course of immunotherapy. A “TCR-T cell therapy” is a type of cellular therapy wherein at least one recombinant TCR sequence is engineered into autologous or allogeneic T cells, and then the engineered TCR-T cells are injected into a patient. In such applications, the TCR is directed against a peptide:MHC of therapeutic interest, for example, a tumor-specific peptide:MHC.

### 9. OTHER INTERPRETATIONAL CONVENTIONS

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

### 10. NUCLEIC ACIDS

In one aspect, the present invention provides isolated nucleic acid molecules. The nucleic acids comprise, for example, polynucleotides that encode all or part of a TCR, for example, one or both chains of a TCR of the invention, or a fragment, derivative, mutein, or variant thereof.

In another aspect, the present invention provides methods to generate libraries of nucleic acids that encode for libraries of TCRs, derived from primary T cells. These libraries of nucleic acids are generated by isolating T cells into single-cell reaction containers, wherein they are lysed and TCR-specific nucleic acids are purified or captured, for example on solid supports such as beads. The present invention provides methods for performing capture of transcripts from millions of single T cells in parallel. Capture of transcripts is followed by amplification of nucleic acids that encode TCR alpha and beta, and subsequent linkage of said nucleic acids into libraries of fused constructs that encode both TCR alpha and beta. In such libraries the native pairing of TCR alpha and beta, as originally found in the input T cells, is maintained. Such methods are performed in parallel on millions of single T cells, such that the resulting library of fused TCR alpha and beta nucleic acids comprises natively paired sequences for millions of single cells.

### 11. EXPRESSION VECTORS

The present invention provides vectors comprising a nucleic acid encoding a polypeptide of the invention or a portion thereof. Examples of vectors include, but are not limited to, plasmids, viral vectors, non-episomal mammalian vectors and expression vectors, for example, recombinant expression vectors.

In another aspect of the present invention, expression vectors containing the nucleic acid molecules and polynucleotides of the present invention are also provided, and host cells transformed with such vectors, and methods of producing the polypeptides are also provided. The term “expression vector” refers to a plasmid, phage, virus or vector for expressing a polypeptide from a polynucleotide sequence. Vectors for the expression of the polypeptides contain at a minimum sequences required for vector propagation and for expression of the cloned insert. An expression vector comprises a transcriptional unit comprising an assembly of (1) a genetic element or elements having a regulatory role in gene expression, for example, promoters or enhancers, (2) a sequence that encodes polypeptides and proteins to be transcribed into mRNA and translated into protein, and (3) appropriate transcription initiation and termination sequences. These sequences may further include a selection marker. Vectors suitable for expression in host cells are readily available and the nucleic acid molecules are inserted into the vectors using standard recombinant DNA techniques. Such vectors can include promoters which function in specific tissues, and viral vectors for the expression of polypeptides in targeted human or animal cells.

The recombinant expression vectors of the invention can comprise a nucleic acid of the invention in a form suitable for expression of the nucleic acid in a host cell. The recombinant expression vectors include one or more regulatory sequences, selected on the basis of the host cells to be used for expression, which is operably linked to the nucleic acid sequence to be expressed. Regulatory sequences include those that direct constitutive expression of a nucleotide sequence in many types of host cells (e.g., SV40 early gene enhancer, Rous sarcoma virus promoter and cytomegalovirus promoter), those that direct expression of the nucleotide sequence only in certain host cells (e.g., tissue-specific regulatory sequences, see Voss et al., 1986, Trends Biochem. Sci. 11:287, Maniatis et al., 1987, Science 236:1237, incorporated by reference herein in their entireties), and those that direct inducible expression of a nucleotide sequence in response to particular treatment or condition (e.g., the metallothionin promoter in mammalian cells and the tet-responsive and/or streptomycin responsive promoter in both prokaryotic and eukaryotic systems (see id.). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The invention further provides methods of making polypeptides. A variety of other expression/host systems may be utilized. Vector DNA can be introduced into prokaryotic or eukaryotic systems via conventional transformation or transfection techniques. These systems include but are not limited to microorganisms such as bacteria (for example, E. coli) transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Mammalian cells useful in recombinant protein production include but are not limited to primary T cells, Jurkat cells, VERO cells, HeLa cells, Chinese hamster ovary (CHO) cell lines, or their derivatives such as Veggie CHO and related cell lines which grow in serum-free media (see Rasmussen et al., 1998, Cytotechnology 28:31) or CHO strain DX-B11, which is deficient in DHFR (see Urlaub et al., 1980, Proc. Natl. Acad. Sci. USA 77:4216-20) COS cells such as the COS-7 line of monkey kidney cells (ATCC CRL 1651) (see Gluzman et al., 1981, Cell 23:175), W138, BHK, HepG2, 3T3 (ATCC CCL 163), RIN, MDCK, A549, PC12, K562, L cells, C127 cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells, or any kind of primary cells, such as T cells Mammalian expression allows for the production of secreted or soluble polypeptides which may be recovered from the growth medium, or expression on the cell surface.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Once such cells are transformed with vectors that contain selectable markers as well as the desired expression cassette, the cells can be allowed to grow in an enriched media before they are switched to selective media, for example. The selectable marker is designed to allow growth and recovery of cells that successfully express the introduced sequences. Resistant clumps of stably transformed cells can be proliferated using tissue culture techniques appropriate to the cell line employed. An overview of expression of recombinant proteins is found in Methods of Enzymology, v. 185, Goeddell, D. V., ed., Academic Press (1990). Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die), among other methods. The transformed cells can be cultured under conditions that promote expression of the polypeptide.

In addition, the polypeptides can be synthesized in solution or on a solid support in accordance with conventional techniques. Various automatic synthesizers are commercially available and can be used in accordance with known protocols. See, for example, Stewart and Young, Solid Phase Peptide Synthesis, 2d. Ed., Pierce Chemical Co. (1984); Tam et al., J Am Chem Soc, 105:6442, (1983); Merrifield, Science 232:341-347 (1986); Barany and Merrifield, The Peptides, Gross and Meienhofer, eds, Academic Press, New York, 1-284; Barany et al., Int J Pep Protein Res, 30:705-739 (1987).

The polypeptides and proteins of the present invention can be purified according to protein purification techniques well known to those of skill in the art. These techniques involve, at one level, the crude fractionation of the proteinaceous and non-proteinaceous fractions. Having separated the peptide polypeptides from other proteins, the peptide or polypeptide of interest can be further purified using chromatographic and electrophoretic techniques to achieve partial or complete purification (or purification to homogeneity). The term “purified polypeptide” as used herein, is intended to refer to a composition, isolatable from other components, wherein the polypeptide is purified to any degree relative to its naturally-obtainable state. A purified polypeptide therefore also refers to a polypeptide that is free from the environment in which it may naturally occur. Generally, “purified” will refer to a polypeptide composition that has been subjected to fractionation to remove various other components, and which composition substantially retains its expressed biological activity. Where the term “substantially purified” is used, this designation will refer to a peptide or polypeptide composition in which the polypeptide or peptide forms the major component of the composition, such as constituting about 50%, about 60%, about 70%, about 80%, about 85%, or about 90% or more of the proteins in the composition.

In some aspects, the present invention includes libraries of TCR-encoding nucleic acid vectors for integration into mammalian genomes. Such vectors include plasmids, retroviruses, and lentivirus. The libraries of nucleic acid vectors may include 10, 100, 1,000, 10,000, or more than 100,000 different TCR-encoding sequences. The sequences are derived from T cells. These libraries of nucleic acids are generated by isolating T cells into single-cell reaction containers, wherein they are lysed and TCR-specific nucleic acids are purified or captured, for example on solid supports such as beads. The present invention provides methods for performing capture of transcripts from millions of single T cells in parallel. Capture of transcripts is followed by amplification of nucleic acids that encode TCR alpha and beta, and subsequent linkage of said nucleic acids into libraries of fused constructs that encode both TCR alpha and beta. In such libraries the native pairing of TCR alpha and beta, as originally found in the input T cells, is maintained. Such methods are performed in parallel on millions of single T cells, such that the resulting library of fused TCR alpha and beta nucleic acids comprises natively paired sequences for millions of single cells. These paired fused amplicons are then engineered into full-length TCR constructs using Gibson Assembly, restriction endonucleases, or other recombinant DNA techniques.

Engineering into full-length TCR constructs is performed on the full library en masse, such that the TCR sequence content and TCR sequence counts of the library are essentially maintained throughout the process. In some aspects, the library of expression vectors is engineered in two steps, such that the TCR fragment amplicon is subcloned into an intermediate vector, and then a second round of Gibson Assembly, restriction digestion, or other recombinant technique is used to engineer additional domains of the TCR into the linker of the TCR fragment amplicon. The native pairing of TCR alpha and beta is essentially maintained throughout the process of engineering into full-length expression vector libraries. The vectors are designed in various orientations, for example, two separate promoters drive expression of TCR alpha and beta, or one promoter drives expression of both TCR alpha and beta, and a translational skip motif is used to separately translate the TCR alpha and beta into separate polypeptides. In some embodiments, the expression vectors comprise sequences for site-directed integration into mammalian production cells, for example, CRISPR-Cas9, Flp-In, Cre/Lox, or zinc finger recombination methods. Site-directed integration ensures that each mammalian production cell encodes a single TCR alpha and beta sequence, and decreases variability in expression levels between single production cells.

### 12. T CELL RECEPTORS

Native TCRs exist in heterodimeric αβ or γδ forms. However, recombinant TCRs consisting of αα or ββ homodimers have previously been shown to bind to peptide MHC molecules. Therefore, the TCR of the invention may be a heterodimeric β TCR or may be an αα or ββ homodimeric TCR. For use in adoptive cell therapy, an b β heterodimeric TCR may, for example, be transfected as full-length chains having both cytoplasmic and transmembrane domains. In certain embodiments TCRs of the invention may have an introduced disulfide bond between residues of the respective constant domains, as described, for example, in WO 2006/000830. TCRs of the invention, particularly αβ heterodimeric TCRs, may comprise an α chain TRAC constant domain sequence and/or a β chain TRBC1 or TRBC2 constant domain sequence. The alpha and beta chain constant domain sequences may be modified by truncation or substitution to delete the native disulfide bond between Cys4 of exon 2 of TRAC and Cys2 of exon 2 of TRBC1 or TRBC2. The alpha and/or beta chain constant domain sequence(s) may also be modified by substitution of cysteine residues for Thr 48 of TRAC and Ser 57 of TRBC1 or TRBC2, the said cysteines forming a disulfide bond between the alpha and beta constant domains of the TCR. TCRs of the invention may be in single chain format, for example see WO 2004/033685. In certain embodiments single chain TCRs of the invention may have an introduced disulfide bond between residues of the respective constant domains, as described in WO 2004/033685.

The invention also provides a cell harbouring a vector of the invention, preferably a TCR expression vector. The vector may comprise nucleic acid of the invention encoding in a single open reading frame, or two distinct open reading frames, the alpha chain and the beta chain respectively. Another aspect provides a cell harbouring a first expression vector which comprises nucleic acid encoding the alpha chain of a TCR of the invention, and a second expression vector which comprises nucleic acid encoding the beta chain of a TCR of the invention. Such cells are particularly useful in adoptive TCR-T or other cell therapy. The cells may be isolated and/or recombinant and/or nonnaturally occurring and/or engineered.

Since the TCRs of the invention have utility in adoptive TCR-T therapy, the invention includes a nonnaturally occurring and/or purified and/or or engineered cell, especially a T cell, presenting a TCR of the invention. There are a number of methods suitable for the transfection of T cells with nucleic acid (such as DNA, cDNA or RNA) encoding the TCRs of the invention (see for example Robbins et al., (2008) J Immunol. 180: 6116-6131). T cells expressing the TCRs of the invention will be suitable for use in adoptive therapy-based treatment of cancers such as those of the pancreas and liver. As will be known to those skilled in the art, there are a number of suitable methods by which adoptive therapy can be carried out (see for example Rosenberg et al., (2008) Nat Rev Cancer 8(4): 299-308). TCR-T cells can be either derived from T cells in a patient's own blood (autologous) or derived from the T cells of another healthy donor (allogenic).

As is well-known in the art TCRs of the invention may be subject to post-translational modifications when expressed by transfected cells. Glycosylation is one such modification, which comprises the covalent attachment of oligosaccharide moieties to defined amino acids in the TCR chain. For example, asparagine residues, or serine/threonine residues are well-known locations for oligosaccharide attachment. The glycosylation status of a particular protein depends on a number of factors, including protein sequence, protein conformation and the availability of certain enzymes. Furthermore, glycosylation status (i.e oligosaccharide type, covalent linkage and total number of attachments) can influence protein function. Therefore, when producing recombinant proteins, controlling glycosylation is often desirable. Glycosylation of transfected TCRs may be controlled by mutations of the transfected gene (Kuball J et al. (2009), J Exp Med 206(2):463-475). Such mutations are also encompassed in this invention.

Certain TCRs of the invention may be in soluble form (i.e. having no transmembrane or cytoplasmic domains). For stability, TCRs of the invention, and preferably soluble 13 heterodimeric TCRs, may have an introduced disulfide bond between residues of the respective constant domains, as described, for example, in WO 03/020763. Some soluble TCRs of the invention are useful for making fusion proteins which can be used for delivering detectable labels or therapeutic agents to antigen presenting cells and tissues containing antigen presenting cells. They may therefore be associated (covalently or otherwise) with a detectable label (for diagnostic purposes wherein the TCR is used to detect the presence of cells presenting peptide:MHC; a therapeutic agent; or a pharmacokinetics-modifying moiety (for example by PEGylation). Detectable labels for diagnostic purposes include for instance, fluorescent labels, radiolabels, enzymes, nucleic acid probes and contrast reagents.

TCRs can be purified from host cells that have been transfected by a gene encoding the TCRs by elution of filtered supernatant of host cell culture fluid using a Heparin HP column, using a salt gradient, or other methods. Fragments or analogs of TCRs can be readily prepared by those of ordinary skill in the art following the teachings of this specification and using techniques well-known in the art. Preferred amino- and carboxy-termini of fragments or analogs occur near boundaries of functional domains Structural and functional domains can be identified by comparison of the nucleotide and/or amino acid sequence data to public or proprietary sequence databases. Computerized comparison methods can be used to identify sequence motifs or predicted protein conformation domains that occur in other proteins of known structure and/or function. Methods to identify protein sequences that fold into a known three-dimensional structure are known.

In certain embodiments, a TCR comprises one or more water soluble polymer attachments, including, but not limited to, polyethylene glycol, polyoxyethylene glycol, or polypropylene glycol. See, e.g., U.S. Pat. Nos. 4,640,835, 4,496,689, 4,301,144, 4,670,417, 4,791,192 and 4,179,337. In certain embodiments, a derivative binding agent comprises one or more of monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers. In certain embodiments, one or more water-soluble polymer is randomly attached to one or more side chains. In certain embodiments, PEG can act to improve the therapeutic capacity for a binding agent, such as a TCR. Certain such methods are discussed, for example, in U.S. Pat. No. 6,133,426, which is hereby incorporated by reference for any purpose.

### 13. METHODS OF IDENTIFYING T CELL RECEPTORS

The variable region of TCRα and δ chains is encoded by a number of variable (V) and joining (J) genes, while TCRβ and γ chains are additionally encoded by diversity (D) genes. During VDJ recombination, one random allele of each gene segment is recombined with the others to form a functional variable region. Recombination of the variable region with a constant gene segment results in a functional TCR chain transcript. Additionally, random nucleotides are added and/or deleted at the junction sites between the gene segments. This process leads to strong combinatorial (depending on which gene regions will recombine) and junctional diversity (which and how many nucleotides will be added/deleted), resulting in a large and highly variable TCR repertoire, which will ensure the identification of a plethora of antigens. Additional diversity is achieved by the pairing of α and β or γ and δ chains to form a functional TCR.

VDJ recombination of the different TCR genes could theoretically generate between 10¹⁵ and 10²⁰ TCR chains. The actual diversity present in a human body is estimated at around 10′³ different clonotypes, implying that the afore-described seemingly random TCR development is obviously not random at all and is subject to different constraints. Moreover, while there are TCRs that are common in the general population, recent high-resolution studies have shown that the majority of TCRs is rare (in analogy to common vs. rare genomic variants). This is one of the reasons why precise methods are necessary to properly investigate complete individual immune repertoires.

Antibody discovery faces many of the same challenges as TCR discovery, but antibody discovery is far more technologically advanced than TCR discovery. For example, methods such as mouse hybridomas (Köhler & Milstein, Nature, 1975, 256(5517):495-7) and phage display (McCafferty et al., Nature, 1990, 348(6301):552-4) are widely used to quickly identify specific and efficacious antibody candidates. Though pioneering groups have described methods for yeast display of TCRs (Kieke et al., PNAS, 1999, 96(10):5651-6), such methods require artificial mutation of natural TCRs, which confounds broader utility. Additionally, TCRs are best studied in the context of T cell surface co-receptors, such as CD8 and CD3 (Kuhns et al., Immunity, 2006, 24(2):133-9). Thus, other groups have reported recombinant expression of TCR libraries in mammalian cells (Chervin et al., Journal of Immunological Methods, 2008, 339(2):175-84; Malecek et al., Journal Immunological Methods, 2013, 392(1-2):1-11). However, reported technologies fail to leverage the TCR diversity of natural human repertoires.

Recently, several groups have described methods that combine microfluidics, multiplex PCR, yeast display, and deep sequencing for ultra-high-throughput discovery of antibodies from human repertoires (Adler et al., MAbs, 2017, 9(8):1282-1296; Wang et al., Nature Biotechnol, 2018, 36(2):152-155). Here, we report a similar technology for ultra-high-throughput discovery of TCRs from human repertoires. Human repertoires can be virus-positive, healthy donors, cancer donors, donors with autoimmunity, or donors with any disease condition. Because the diversity of TCRαβ repertoires are generated by millions of single cells expressing different TCRα and TCRβ sequences, we first use a droplet microfluidic technology to isolate single cells into droplet emulsions and natively pair TCRα and TCRβ on a single cell level. The microfluidic technology can process millions of single T cells in an hour, which is significantly higher throughput than previously reported methods for TCRαβ pairing (Turchaninova et al., Eur Journal of Immunology 2013, 43(9):2507-15; Howie et al., Science Translational Medicine 2015, 7(301):301ra131). TCRα and TCRβ transcripts are captured from lysed single cells, amplified, and then physically linked into a single amplicon for subsequent cloning into expression vectors. Lysis and amplification are performed in two steps, since the reagents for lysis are incompatible with efficient RT-PCR.

Some other methods are available for natively pairing TCRα and TCRβ via a single cell barcoding method, for example through a commercial group (10× Genomics; Azizi et al., Cell 2018, 174(5):1293-1308.e36). Single cells are isolated into microfluidic droplets with molecular barcodes, and then TCRα and TCRβ from the single cells are fused to the unique barcodes. The single cell TCRα and TCRβ pairing is then inferred through bioinformatics. Though these molecular identifiers might offer advantages in terms of quantification, as used elsewhere for methods that do not leverage single cells (Shugay et al., 2014), single cell barcoding methods do not generate libraries of physically linked TCRα and TCRβ. This complicates any downstream efforts to identify binding and avidity properties of the TCRαβ sequences.

In our method, the library of millions of physically linked, natively paired TCRαβ amplicons is cloned en masse into expression vectors. The vectors are then subjected to restriction digestion en masse, and a DNA insert that encodes a TCR constant domain and translational skip sequence is cloned into the library. The full-length TCRαβ libraries are then packaged into lentiviral constructs and transduced into Jurkat cells that lack endogenous TCRβ expression and which are additionally engineered to stably express CD8. The resulting TCR-Jurkat libraries comprise natively linked TCRαβ sequences from millions of single T cells. The TCR-Jurkat libraries are immortal and renewable, enabling multiple rounds of panning with multiple antigens, using both binding to MHC multimers and activation by artificial antigen-presenting cells (aAPCs).

Similar methods can be applied to T cell repertoires from any animal with T cells, for example, mouse, rat, dog, cow, rabbit, or horse.

### 14. SEQUENCES

SEQ ID NOS 1-12 as found in the sequence listing submitted with this application, recite the pairs of sequences of the TCR α and β V(D)J polypeptides for TCR clones 1-6. For example, TCR clone 1 comprises SEQ ID NO:1 (TCRα for TCR 1) and SEQ ID NO:2 (TCRβ for TCR 1), TCR clone 2 comprises SEQ ID NO:3 (TCRα for TCR 2) and SEQ ID NO:4 (TCRβ for TCR 2), TCR clone 3 comprises SEQ ID NO:5 (TCRα for TCR 3) and SEQ IDNO:6 (TCRβ for TCR 3), and so on.

SEQ ID NOS: 13-790940 recite the pairs of CDR3 alpha sequences and CDR3 beta sequences for TCR clones 7-395464. For example, TCR clone 7 comprises the CDR3 alpha sequence of SEQ ID NO:13 and the CDR3 beta sequence of SEQ ID NO:14. Six of these clones are duplicates of TCR clones 1-6.

SEQ ID NOS:790941-791100 are the sequences of the TCR α and β V(D)J polypeptides.

SEQ ID NO:791101 is the amino acid sequence of the HLA_A 0201 gp100 peptide.

| SEQ ID NOS | Description |
|---|---|
| 1, 3, 5, 7, 9, 11 | Jurkat-TIL full length TCR-alpha |
| | variable sequences |
| 2, 4, 6, 8, 10, 12 | Jurkat-TIL full length TCR-beta |
| | variable sequences |
| 13, 15, 17, through 790939 | Jurkat-TIL CDR3-alpha sequences |
| (odd numbers) | |
| 14, 16, 18, through 790940 | Jurkat-TIL CDR3-beta sequences |
| (even numbers) | |
| 790941-790983 | TCR-alpha V gene sequences |
| 790984-791035 | TCR-alpha J gene sequences |
| 791036-791086 | TCR-beta V gene sequences |
| 791087-791100 | TCR-beta J gene sequences |
| 791101 | HLA_A 0201 gp100 peptide |

In some embodiments, disclosed is an isolated, non-naturally occurring mammalian cell comprising recombinant T cell receptor (TCR) that specifically binds gp100 antigen HLA*A 02:01/gp100 (KTWGQYWQV) (SEQ ID NO:791101), wherein the TCR comprises an alpha chain and a beta chain, the alpha chain comprising an alpha variable domain comprising an alpha CDR3 and the beta chain comprising a beta variable domain comprising an beta CDR3, wherein the alpha CDR3 sequence is selected from SEQ ID NOS: 13-790939, odd numbers, and the beta CDR3 sequence is selected from SEQ ID NOS: 14-790940, even numbers. In some embodiments, the alpha variable domain comprises a sequence selected from SEQ ID NOS: 1-11, odd numbers, and the beta variable domain comprises a sequence selected from SEQ ID NOS: 2-12, even numbers.

### 15. PHARMACEUTICAL COMPOSITIONS

For administration to patients, the TCRs or TCR-T cells of the invention may be provided in a pharmaceutical composition together with one or more pharmaceutically acceptable carriers or excipients. TCR-T cells in accordance with the invention will usually be supplied as part of a sterile, pharmaceutical composition which will normally include a pharmaceutically acceptable carrier. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms. The pharmaceutical composition may be adapted for administration by any appropriate route, preferably a parenteral (including subcutaneous, intramuscular, or preferably intravenous) route. Such compositions may be prepared by any method known in the art of pharmacy, for example by mixing the active ingredient with the carriers) or excipient(s) under sterile conditions. TCRs, pharmaceutical compositions, vectors, nucleic acids and cells of the invention may be provided in substantially pure form, for example at least 50%, at least 60%, at least 70%, at least 80%, at least 85%, at least 90%, at least 91%, at least 92%, at least 93%, at least 94%, at least 95%, at least 96%, at least 97%, at least 98%, at least 99% or 100% pure.

Also provided by the invention are: (i) a non-naturally occurring and/or purified and/or engineered TCR which binds the target peptide:MHC, or a cell expressing and/or presenting such a TCR, for use in medicine, preferably in a method of treating cancer. The method may comprise adoptive therapy; (ii) the use of a TCR which binds the target peptide:MHC, or a cell expressing and/or presenting such a TCR, in the manufacture of a medicament for treating cancer; (iii) a method of treating cancer in a patient, comprising administering to the patient a TCR which binds the peptide:MHC target, or a cell expressing and/or presenting such a TCR. Therapeutic agents which may be associated with the TCRs of the invention include immunomodulators, radioactive compounds, enzymes (perforin for example) or chemotherapeutic agents (cis-platin for example). To ensure that toxic effects are exercised in the desired location the toxin could be inside a liposome linked to a TCR so that the compound is released slowly. This will prevent damaging effects during the transport in the body and ensure that the toxin has maximum effect after binding of the TCR to the relevant antigen presenting cells.

Other suitable therapeutic agents include small molecule cytotoxic agents, i.e. compounds with the ability to kill mammalian cells having a molecular weight of less than 700 Daltons. Such compounds could also contain toxic metals capable of having a cytotoxic effect. Furthermore, it is to be understood that these small molecule cytotoxic agents also include pro-drugs, i.e. compounds that decay or are converted under physiological conditions to release cytotoxic agents. Examples of such agents include cis-platin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetreate glucuronate, auristatin E vincristine and doxorubicin; peptide cytotoxins, i.e. proteins or fragments thereof with the ability to kill mammalian cells. For example, ricin, diphtheria toxin, pseudomonas bacterial exotoxin A, DNase and RNase; radio-nuclides, i.e. unstable isotopes of elements which decay with the concurrent emission of one or more of a orb particles, or g rays. For example, iodine 131, rhenium 186, indium 111, yttrium 90, bismuth 210 and 213, actinium 225 and astatine 213; chelating agents may be used to facilitate the association of these radio-nuclides to the high affinity TCRs, or multimers thereof; immuno-stimulants, i.e. immune effector molecules which stimulate immune response. For example, cytokines such as IL-2 and IFN-g, Superantigens and mutants thereof; TCR-HLA fusions; chemokines such as IL-8, platelet factor 4, melanoma growth stimulatory protein, etc; antibodies or fragments thereof, including anti-T cell or NK cell determinant antibodies (e.g. anti-CD3, anti-CD28 or anti-CD16); alternative protein scaffolds with antibody like binding characteristics complement activators; xenogeneic protein domains, allogeneic protein domains, viral/bacterial protein domains, viral/bacterial peptides.

### 16. METHODS OF USE

Therapeutic TCRs may be used that specifically bind to antigen target or targets.

In vivo and/or in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. Effective doses may be extrapolated from dose-response curves derived from in vitro or animal model test systems.

An oligopeptide or polypeptide is within the scope of the invention if it has an amino acid sequence that is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to least one of the CDRs provided herein.

The terms “treatment,” “treating,” and the like are used herein to generally mean obtaining a desired pharmacologic and/or physiologic effect. The effect may be prophylactic, in terms of completely or partially preventing a disease, condition, or symptoms thereof, and/or may be therapeutic in terms of a partial or complete cure for a disease or condition and/or adverse effect, such as a symptom, attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms). Improvements in any conditions can be readily assessed according to standard methods and techniques known in the art. The population of subjects treated by the method of the disease includes subjects suffering from the undesirable condition or disease, as well as subjects at risk for development of the condition or disease.

By the term “therapeutically effective dose” or “effective amount” is meant a dose or amount that produces the desired effect for which it is administered. The exact dose or amount will depend on the purpose of the treatment, and will be ascertainable by one skilled in the art using known techniques (see, e.g., Lloyd (1999) The Art, Science and Technology of Pharmaceutical Compounding).

The term “sufficient amount” means an amount sufficient to produce a desired effect.

The term “therapeutically effective amount” is an amount that is effective to ameliorate a symptom of a disease. A therapeutically effective amount can be a “prophylactically effective amount” as prophylaxis can be considered therapy.

The term “ameliorating” refers to any therapeutically beneficial result in the treatment of a disease state, e.g., a neurodegenerative disease state, including prophylaxis, lessening in the severity or progression, remission, or cure thereof.

The actual amount administered, and rate and time-course of administration, will depend on the nature and severity of protein aggregation disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

In some embodiments, the pharmaceutical composition is administered by inhalation, orally, by buccal administration, by sublingual administration, by injection or by topical application.

In some embodiments, the pharmaceutical composition is administered in an amount sufficient to modulate survival of neurons or dopamine release. In some embodiments, the major cannabinoid is administered in an amount less than 1 g, less than 500 mg, less than 100 mg, less than 10 mg per dose.

In some embodiments, the pharmaceutical composition is administered once a day, 2-4 times a day, 2-4 times a week, once a week, or once every two weeks.

### 17. EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The following publication is incorporated by reference for all purposes: Spindler, M. J., Nelson, A. L., Wagner, E. K. et al. Massively parallel interrogation and mining of natively paired human TCRαβ repertoires. Nat Biotechnol (2020). https://doi.org/10.1038/s41587-020-0438-y.

### Example 1: Identification of Anti-Gp100 TCRs from Human Donor T Cell Repertoires

Sourcing and Processing Human Materials

Human TILs were generated from tumor biopsies from consenting individuals undergoing TIL clinical therapy for metastatic melanoma using a proprietary method which in brief involves the following. Tumor biopsies were disaggregated to a single cell suspension using collagenase and DNAse, then plated in tissue culture plates in the presence of IL-2 for up to 21 days. Following this initial outgrowth the TIL underwent a rapid expansion protocol (REP) by mixing the derived TIL with donor-mixed irradiated PBMC feeder cells with addition of OKT3 and IL-2, for 14 days prior to cryopreservation.

### Generating Paired TCRα-TCRβ Linkage Libraries

Library generation is divided into three steps: (i) poly(A)+ mRNA capture, (ii) multiplexed overlap extension reverse transcriptase polymerase chain reaction (OE-RT-PCR), and (iii) nested PCR to remove artifacts and add adapters for deep sequencing or expression libraries (Adler et al., 2017).

For poly(A)+ mRNA capture, we used a custom designed co-flow emulsion droplet microfluidic chip fabricated from glass (Dolomite). The microfluidic chip has two input channels for fluorocarbon oil (Dolomite), one input channel for the cell suspension mix, and one input channel for oligo-dT beads (New England Biolabs) in 0.5M NaCl, 0.5% Tween-20, and 20 mM DTT. The input channels are etched to 50 μm×150 μm for most of the chip's length, narrow to 55 μm at the droplet junction, and are coated with hydrophobic Pico-Glide (Dolomite). Three Mitos P-Pump pressure pumps (Dolomite) are used to pump the liquids through the chip. Droplet size depends on pressure, but typically we find that droplets of ˜45 μm diameter are optimally stable. Emulsions were collected into 1.5 mL microcentrifuge tubes and incubated at 40° C. for 30 minutes to capture mRNA onto oligo-dT beads. Emulsions were then broken using Pico-Break (Dolomite) and mRNA-bound beads are magnetically isolated.

For multiplex OE-RT-PCR, mRNA-bound beads were re-encasuplated into droplets with an OE-RT-PCR mix. The OE-RT-PCR mix contains 2× one step RT-PCR buffer (ThermoFisher), 2.0 mM MgSO₄, SuperScript III reverse transcriptase (ThermoFisher), and Platinum Taq (ThermoFisher), plus a mixture of primers directed against the TRAC, TRBC, and all V-gene regions. TCRa and TCRb chains are physically linked by overlapping primer sequences included on the TRAC and TRBV primers. The amplified DNA was recovered from the droplets using a proprietary droplet breaking solution (GigaMune) and purified using a QIAquick PCR Purification Kit (Qiagen).

For nested PCR, the OE-RT-PCR product was first run on a 1.7% agarose gel and a band at 800-1200 bp was excised and purified using NucleoSpin Gel and PCR Clean-up Kit (Macherey-Nagel). Nested PCR was performed using NEBNext amplification mix (NEB) to add adapters for Illumina sequencing or cloning into a mammalian expression construct. PCR products were run on a 1.2% agarose gel, and the 800-1100 bp band was excised and purified using NucleoSpin Gel and PCR Clean-up Kit (Macherey-Nagel).

### Linked TCRαβ Repertoire Sequencing

Deep TCRαβ sequencing libraries were quantified using a quantitative PCR Illumina Library Quantification Kit (Kapa Biosystems) and diluted to 8.5-10 pM. Libraries were sequenced on a MiSeq (Illumina) using v3 600-cycle MiSeq Reagent Kits, according to the manufacturer's instructions. To identify the paired sequences from the TCRαβ libraries, we obtain forward reads of 357 cycles that cover the TCRα V gene and CDR3, and reverse reads of 162 cycles that cover the TCRβ CDR3 and enough of the TCRβ V gene for accurate calling.

To remove base call errors, we use the expected error filtering method of Edgar and Flyvbjerg (Edgar, Bioinformatics 2015, 31(21):3476-82). The expected number of errors (E) for a read is calculated from its Phred scores. By default, reads with E>1 are discarded, leaving reads for which the most probable number of base call errors is zero. For the clonotypes analyzed in this study, we excluded singletons and required that unique CDR3α+CDR3β paired sequences share the same V and J genes.

To identify reading frame and CDR3 amino acid sequences generated by V(D)J rearrangements, we first processed a database of well-curated TCR sequences (IMGT, http://www.imgt.org/download/LIGM-DB/; Lefranc et al., 2009) to generate position-specific sequence matrices (PSSMs) for the 5′ and 3′ CDR3 junctions. Each nucleotide sequence from the Illumina sequencing runs was translated into all reading frames. We then used the PSSMs to identify the FR3-CDR3 (5′) and CDR3-FR4 (3′) junctions and the appropriate protein reading frame for each of the nucleotide sequences. To report a CDR3 sequence, we required 5′ and 3′ PSSM hits in the same reading frame. Additionally, sequences that had low PSSM identity scores were marked with an exclamation point. These steps allowed us to predict valid, functional, CDR3 sequences with high confidence. We queried TCRα and TCRβ nucleotide sequences against the IMGT database of reference V and J gene germline sequences using UBLAST (https://www.drive5.com/usearch/manual/ublast_algo.html; Edgar, 2010); V and J genes were identified based on the UBLAST alignments with the best alignment (lowest E-values).

### Single Chain TCRα and TCRβ Repertoire Sequencing

For single chain sequencing of the recombinant TCRα-TCRβ expression libraries, TCRα and TCRβ V(D)J regions were amplified separately using universal primers, that contained adapters for Illumina sequencing, within the TRAV.SS and TRAC regions for TCRα and within the TRBV.SS and TRBC regions for TCRβ. We conducted RT-PCR off RNA samples using SuperScript III reverse transcriptase (ThermoFisher) and Platinum Taq (ThermoFisher). We used NEBNext amplification mix (NEB) for PCR off plasmid DNA. These amplicons were run on 1.7% agarose gels and the 500-600 bp band was excised and purified using NucleoSpin Gel and PCR Clean-up Kit (Macherey-Nagel). Samples were separately quantified using an Illumina Library Quantification Kit (Kapa Biosystems). After diluting to 9 pM, libraries were sequenced on a MiSeq (Illumina) using v2 500-cycle MiSeq Reagent Kits, according to the manufacturer's instructions. We obtained overlapping forward and reverse reads of 255 cycles for TCRα and TCRβ separately. TCRα and TCRβ sequences were analyzed separately using the same methods as described above for paired sequences, with the additional requirement that overlapping reads were observed.

### Generating Recombinant TCRα 0 Jurkat Expression Libraries

We developed a subcloning workflow to convert the linked TCRαβ amplicons into full-length lentiviral expression constructs. In this workflow, we first used nested PCR to add overhang adapters to the 5′ and 3′ ends of the linked TCRαβ amplicons for downstream Gibson assembly. Then, we used NEBuilder HiFi DNA Assembly Master Mix (New England Biolabs) to insert the linked TCRαβ library into a pReceiver-based lentiviral vector (GeneCopoeia) that contains the EF1α promoter, a TCRα signal sequence, the TCRβ constant region, and the Puromycin resistance gene. We transformed this intermediate library into Endura electrocompetent E. coli (Lucigen), plated onto LB Lennox carbenicillin plates (Teknova), and scraped and pooled >5 million colonies from each library for plasmid purification. Plasmids were purified using the endotoxin free ZymoPURE II Plasmid Maxiprep Kit (Zymo Research). These intermediate libraries were linearized with a NheI-HF (New England Biolabs) restriction digest present within the linker region, run on a 0.8% agarose gel, and gel extracted using the NucleoSpin Gel and PCR Clean-up Kit (Macherey-Nagel). To create the full-length TCRαβ lentiviral libraries, we performed a second Gibson assembly to insert the TCRα constant region, a ribosomal skip motif (P2A; Funston, Journal General Virology 2008, 89(Pt 2):389-96), and a TCRβ signal sequence. These full-length TCRαβ lentiviral libraries were transformed into Endura electrocompetent cells and purified using the endotoxin free maxiprep kit as described above.

### Lentiviral Transduction of Jurkat Cells

We then optimized our lentiviral transduction protocol to obtain a low transduction efficiency to ensure that we expressed only one TCRαβ pair per cell. We packaged lentivirus into VSV-G pseudotyped lentiviral particles using the 3^{rd} generation ViraSafe Lentiviral Packaging System (Cell Biolabs) and Lenti-Pac 293Ta cells (GeneCopoeia). 21 million Lenti-Pac 293Ta cells per 10 cm plate were transfected with 4.3 ug pCMV-VSV-G, 4.3 ug pRSV-Rev, 4.3 ug pCgpV, and 4.3 ug of the TCRαβ expression libraries using Lipofectamine 3000 (ThermoFisher) following the manufacturer's protocol. Lentiviral supernatant was collected at 48 hours post-transfection, spun down at 500×g for 10 minutes to eliminate cellular debris, and clarified through a 0.45 um syringe filter. RNA was isolated from fresh lentiviral supernatant using the NucleoSpin RNA Virus kit (Macherey-Nagel) following the manufacturer's protocol. Fresh lentiviral supernatant was used to transduce CD8⁺ ΔTCRβ Jurkat cells. Clarified lentiviral supernatant was added at a 1:10 ratio with Jurkat cells in RPMI media with 10% FBS and 8 ug/ml Polybrene (EMD Millipore). Jurkat cells were incubated with lentiviral particles for 6 hours and then media was exchanged. Two days after viral transduction, Jurkat cells were analyzed for cell surface CD3 and TCRαβ expression to measure viral transduction efficiency. Cells were then cultured for 14 days with puromycin to select for stable integration and again assessed for CD3 and TCRαβ surface expression. To generate the natively paired TCRαβ Jurkat expression libraries, we transduced 40 million ΔTCRβ Jurkat and on day 2 observed surface expression on 8-14% of transduced cells compared to 4% on the parental Jurkat cells. TCRαβ surface increased to 42-56% following selection. For monoclonal TCRαβ cell line generation, 800,000 CD8+ΔTCRβ Jurkat cells were transduced and selected with puromycin for 14 days. CD3 and TCRαβ surface expression was measured following selection.

### Screening Recombinant TCRαβ Jurkat Libraries

We screened the TCRαβ Jurkat libraries with peptide-HLA (pHLA) dextramers (Immudex) targeting gp100 (HLA*A 02:01 restricted KTWGQYWQV peptide). We stained 2-5 million CD8+ TCRαβ Jurkat library cells with 10 ul of APC-conjugated dextramer at room temperature for 10 minutes. Cells were then stained with an anti-CD3-FITC antibody (clone: UCHT1; BioLegend) for 30 minutes at 4° C. and DAPI (BioLegend) to assess cell viability. Cells were then sorted on a FACSMelody (BD Biosciences) for Live CD3⁺/dextramer⁺ cells. The sorted Jurkat cells were recovered and expanded in RPMI media with 10% FBS and 100 U/ml Pen/Strep (Gibco). Once cells reached high viability (>85%) and appropriate cell numbers, 2 million cells were lysed, and RNA was extracted using the NucleoSpin RNA Plus kit (Macherey-Nagel) for single chain TCRα and TCRβ repertoire sequencing as described above. Multiple rounds of dextramer staining, FACS sorting, and cell expansion were conducted to enrich for populations of pHLA-binding TCRs.

Following pHLA-binding enrichment, we co-cultured CD8⁺ TCRαβ Jurkat cell populations with peptide-pulsed antigen presenting cells (APCs) to assess cell activation. We pulsed T2 cells with 10 μM peptide, mixed 200,000 peptide-pulsed T2 cells with 200,000 CD8⁺TCRαβ Jurkat cells per well in 96-well round-bottom plates (Falcon), and cultured for 16-20 hours. Cells were harvested out of the round-bottom plates and stained for HLA-A2 (clone: BB7.2; BioLegend), CD69 (clone: FN50; BioLegend), CD62L (clone: DREG-56; Bio-Legend), and cell viability with DAPI. Cells were analyzed on a FACSMelody or CytoFLEX LX (Beckman Coulter) for activation (HLA-A2−/CD69+/CD62L−). We used 1× Cell Stimulation Cocktail (eBioscience, ThermoFisher) as a positive control and irrelevant peptide-pulsed T2 cells as a negative control. To identify TCRs present in peptide-activated Jurkat cells, we co-cultured partially enriched TCRαβ Jurkat cell populations with peptide-pulsed T2 cells, stained with the activation markers described above and sorted for activated (HLA-A2−/CD69+/CD62L−) cells on a FACSMelody. These activated cells were lysed and RNA isolated using the NucleoSpin RNA Plus XS kit (Macherey-Nagel) for single chain TCRα and TCRβ repertoire sequencing. Peptides were synthesized at >90% purity (ELIM Biopharm), resuspended in DMSO to 4 mg/ml, aliquoted for single use, and stored at −20° C.

Therapeutically relevant TCRs should activate T cells upon binding their cognate peptide:MHC, but prior work has established that TCRs can bind their peptide:MHC target but fail to activate T cells (Sibener et al., Cell 2018, 174(3):672-687.e27). Thus, to further reduce false positives, we performed in vitro activation screens on MHC dextramer-enriched TCRαβ Jurkat populations Panning Round 2, 3, or 4 TCR-Jurkat populations were incubated with peptide-pulsed T2 cells, and then FACS-selected for increased cell surface CD69 and decreased CD62L expression. The ratio of the frequency of a TCR in the CD69+/CD62L-fraction to its frequency in the CD69-/CD62L+ fraction was used to quantify the TCR's ability to activate T cells. Activation ratios were integrated with corresponding TCRαβ read frequencies after the 3^{rd} or 4^{th} round of MHC dextramer panning, to assess the likelihood of a true positive.

### Monoclonal TCRαβ Characterization

Enriched TCRα and TCRβ single chain sequences were identified from the pHLA-binding and cell activation screens. We used this enrichment data and the natively paired TCRα-TCRβ sequencing data to identify candidate antigen-reactive TCR clones. We designed full-length TCRα-TCRβ lentiviral expression constructs using the Illumina sequencing data, specifically the CDR3 nucleotide sequences and V-gene calls, and synthesized these plasmids using the BioXp 3200 system (SGI-DNA). These monoclonal TCRαβ expression constructs follow the same layout as the TCRαβ libraries. Lentiviral plasmids were sequence verified by Sanger sequencing, packaged into VSV-G pseudotyped lentiviral particles, transduced into ΔTCRβ Jurkat cells, and stable cell lines were selected.

Monoclonal TCRαβ Jurkat cell lines were assessed for pHLA binding and cellular activation. We stained 0.5-1 million cells with 5 ul of pHLA dextramer and anti-CD3 antibodies as described above. We then ran co-culture assays with the monoclonal CD8⁺TCRαβ Jurkat cell lines that showed pHLA binding. As described above, we pulsed T2 cells with 1004 peptide, mixed 200,000 peptide-pulsed T2 cells with 200,000 TCRαβ Jurkat cells per well, and measured cell activation by staining for CD69 and CD62L.

We then measured functional avidity of the monoclonal TCRs that showed cellular activation using T2 cells pulsed with a dilution series of peptide (1E-7-10 μM). Cells were co-cultured in duplicate wells for 20 hours, harvested, pooled, and stained for activation markers as described above. Cells were run on the CytoFLEX LX and Jurkat cell CD69 Median Fluorescence Intensity (MFI) was calculated using FlowJo (Treestar) and analyzed in Prism (GraphPad). Two independent experiments were conducted, and the mean and standard deviation values were plotted. TCR number is equivalent to TCR clone.

TABLE 4
Funtional annotation of TCR sequences

| | Library | Library | Monoclonal | Monoclonal | TCR |
|---|---|---|---|---|---|
| Target | Binding | Activation | Binding | Activation | number |
| PMEL | Yes | Yes | Yes | Yes | 1 |
| (gp100) | | | | | |
| PMEL | Yes | Yes | Yes | Yes | 2 |
| (gp100) | | | | | |
| PMEL | Yes | Yes | Yes | Yes | 3 |
| (gp100) | | | | | |
| PMEL | Yes | Yes | Yes | Yes | 4 |
| (gp100) | | | | | |
| PMEL | Yes | Yes | Yes | Yes | 5 |
| (gp100) | | | | | |

### 18. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 19. EQUIVALENTS

Whereas various specific embodiments have been illustrated and described, the above specification is not restrictive. It will be appreciated that various changes can be made without departing from the spirit and scope of the invention(s). Many variations will become apparent to those skilled in the art upon review of this specification.

1. A non-naturally occurring mammalian cell, comprising recombinant T cell receptor subunits SEQ ID 1 and SEQ ID 2, that specifically binds gp100 antigen HLA*A 02:01/gp100 (KTWGQYWQV).

2. A non-naturally occurring mammalian cell, comprising recombinant T cell receptor subunits SEQ ID 3 and SEQ ID 4, that specifically binds gp100 antigen HLA*A 02:01/gp100 (KTWGQYWQV).

3. A non-naturally occurring mammalian cell, comprising recombinant T cell receptor subunits SEQ ID 5 and SEQ ID 6, that specifically binds gp100 antigen HLA*A 02:01/gp100 (KTWGQYWQV).

4. A non-naturally occurring mammalian cell, comprising recombinant T cell receptor subunits SEQ ID 7 and SEQ ID 8, that specifically binds gp100 antigen HLA*A 02:01/gp100 (KTWGQYWQV).

5. A non-naturally occurring mammalian cell, comprising recombinant T cell receptor subunits SEQ ID 9 and SEQ ID 10, that specifically binds gp100 antigen HLA*A 02:01/gp100 (KTWGQYWQV).
